(19) United States
(12) Patent Application Publication
(10) Pub. No.: US 2013/0017264 A1
(43) Pub. Date: Jan. 17, 2013

(54) ALGINATE TUBE DRUG DELIVERY SYSTEM AND METHOD THEREFOR

(71) Applicants: Jayant J. KHANDARE, Mumbai (IN); Sanjay P. Boldhane, Mumbai (IN)

(72) Inventors: Jayant J. KHANDARE, Mumbai (IN); Sanjay P. Boldhane, Mumbai (IN)

(73) Assignee: PIRAMAL LIFE SCIENCES LIMITED

(21) Appl. No.: 13/183,598

(22) Filed: Jul. 15, 2011

(51) Int. Cl. A61K 9/00; A61K 47/26; B05D 3/10; A61P 39/00

(52) U.S. Cl. 424/488; 514/779; 427/2.14

(57) ABSTRACT

A drug delivery system which comprises alginate tube that is prepared by coating a substrate with alginate gel. One or more therapeutic drugs may also be present in the alginate gel or in the cavity of the tube. The activity of the alginate drug delivery system is highly adjustable so that the release may be controlled as required. The rate at which the system releases the drug and the concentration of the drug released can be adjusted by varying; the number of layers of the alginate tubes, the number of open or closed ends of the tubes, or the number of tube layers containing the drug.

### FIELD OF THE INVENTION

This invention relates to a drug delivery system and method therefor.

### BACKGROUND OF THE INVENTION

The controlled release of a drug is important in the therapeutic effect of the drug. It is often desired to control the release of an orally ingested drug to improve the bioavailability and therapeutic effect of the drug. In some instances it is desired to increase the gastric retention time of a drug in the gastrointestinal system which often results in improved bioavailability and enhanced therapeutic efficacy of the drug. For instance, many drugs are only efficiently absorbed in the stomach and small intestine. Additionally, certain conditions require local treatment in the upper portion of the gastrointestinal tract. An increase in the duration that a therapeutic drug is in gastrointestinal system will be very beneficial in these circumstances. Furthermore, the necessary level of dosage required for optimal therapeutic effect may be reduced by an increased duration of the drug in the gastrointestinal system including the stomach.

Numerous drug delivery systems have been developed to attempt to increase the duration of an orally administered drug in the gastrointestinal system and control its release. For example, bioadhesive systems, swelling systems, high density systems, and floating systems such as microspheres, granules, capsules, and tablets have been developed. However, many of these systems are unable to deliver an extended retention of the drug within the gastrointestinal system including the stomach in order to maximize the therapeutic benefit of a drug.

Accordingly, an aspect of the present invention is a drug delivery system comprising one or more alginate tubes.

Another aspect of the present invention is an orally-ingested drug delivery system comprising one or more alginate tubes.

Yet another aspect of the present invention is a method of manufacturing a drug delivery system comprising one or more alginate tubes.

Still another aspect of the present invention is the use of the drug delivery system in the treatment of a disorder or a disease or an illness or for prophylactic purposes or both in a subject in need thereof.

### SUMMARY OF THE INVENTION

The present invention is directed to an alginate tube drug delivery system and the use of the system to deliver a drug to a subject. Another aspect of the invention is the use of the alginate tube drug delivery system for the treatment of an illness in a patient and for prophylactic purposes. The present invention is also directed to a method of manufacture of an alginate tube drug delivery system.

### DESCRIPTION OF THE DRAWINGS

FIG. 1 shows a graph comparing the buoyancy and floatability of alginate tubes of the present invention as compared to glass tubes.

FIG. 2 shows a graph comparing Metformin HCl release in various alginate tube drug delivery systems

FIG. 3 shows a graph comparing drug release rates in various alginate tube drug delivery systems.

FIG. 4a shows graphs of Metformin HCl released in coated and uncoated 3-layered alginate tube drug delivery systems.

FIG. 4b shows a chart of Metformin HCl released in coated and uncoated 3-layered Alginate Eudragit tube drug delivery systems.

FIG. 5a shows two different alginate tubes

FIG. 5b shows a graph of the drug release in various alginate tube drug delivery systems

FIG. 6 shows a graph of Metformin HCl release in various alginate tube drug delivery systems.

FIG. 7 shows a graph of drug release in an alginate tube drug delivery system.

FIG. 8 shows a graph comparing the drug release in various alginate tube drug delivery systems.

### DETAILED DESCRIPTION OF THE INVENTION

Before describing the present invention in detail, it has to be understood that this invention is not limited to particular embodiments described in this application. It is also to be understood that the terminology used herein is for the purpose of describing particular embodiments only, and is not intended to be limiting.

As used in the specification and claims, singular terms including, but not limited to, “a”, “an” and “the” include plural references unless the context clearly indicates otherwise. Plural terms include singular references unless the context clearly indicates otherwise. Unless defined otherwise, all technical and scientific terms used herein have the same meaning as commonly understood by one of the ordinary skill in the art to which the invention belongs.

The term “treating”, “treat” or “treatment” as used herein includes preventive (prophylactic) treatment. The term “treating”, “treat” or “treatment” as used herein includes palliative treatment.

By “pharmaceutically acceptable” it is meant the carrier, diluent, excipients, and/or salt must be compatible with the other ingredients of the formulation, and not deleterious to the recipient thereof.

The term “subject” includes living organisms. Non-limiting examples of subjects include humans, monkeys, cows, sheep, goats, dogs, cats, mice, rats, and transgenic species thereof.

The term ‘alginate’ refers to the basic form of alginate biodegradable polymer, while the term ‘alginate mixture’ will refer to a alginate dissolved or suspended or gelled in an aqueous solutions to form a biphasic or mutli-phasic system or even as a gel form with different viscosities at room temperature.

The alginate tube drug delivery system may comprise a single layer of alginate or may be multilayered. Alginate refers to a hydrocolloid consisting of salts of alginic acid. Alginate includes, but is not limited to, salts of alginic acid including, but not limited to, magnesium alginate, sodium alginate, potassium alginate, and calcium alginate or its complexes with other inorganic or organic molecules such as ammonium, propylene glycols and esters etc.

The alginate tube is prepared by preparing alginate gel. A therapeutic drug can be added to the alginate gel. In an embodiment of the invention, the therapeutic drug is dissolved or dispersed in the alginate gel. The tube may be dipped or immersed in the alginate gel or may be exposed to the alginate gel by any other method known in the art. The alginate gel is coated on a substrate and then dipped in salt solution to form an alginate. The substrate coated with the alginate gel may be dipped in a salt solution to form, for example, magnesium alginate, sodium alginate, potassium alginate, calcium alginate, etc. In an embodiment of the invention, the substrate coated with alginate gel is dipped in calcium chloride to form calcium alginate.

The substrate may be any substrate on which the alginate mixture will adhere to and produce the desired shape. Substrates should be preferably smooth, have properties so they do not stick to the alginate gel once the tubes are dried and result in a tube being formed after the substrate is removed from the dried coating. Suitable substrates include but are not limited to glass capillary tubes, thin glass rods, or any metallic or non metallic solid forms, wires or pins.

After the alginate mixture reacts with salt, the alginate solidifies and a coating of alginate is formed over the substrate such that a tube is formed. The tube is dried and if desired, one or more additional layers of alginate gel may be deposited on the outermost layer of the substrate. The tube may be dried at room temperature, by fan, oven or other means known in the art.

The substrate such as a glass capillary tube is exposed to the alginate gel mixture for a few seconds. The substrate may be dipped or immersed in the alginate gel or may be exposed to the alginate gel by any other method known in the art sufficient for coating the substrate with alginate gel. The substrate is subsequently dipped into a 1-10% salt solution for approximately 1-20 minutes to form a divalent complex that is solidified. The coated substrate is air dried for sufficient time period before the substrate is removed to form a tube. As an example, the substrate may be removed from the glass capillary in approximately 10 minutes-24 hrs. The tubes are further air dried for approximately 24-48 hours. Alternatively, the tubes may be dried by fan, oven or other means known in the art. Drying time will depend on the method used to dry the tube. Additional layers are formed by repeating the steps described above.

The tubes can have a concentration of roughly 0.5 mg to 500 mg of therapeutic drug in each tube. The drug may be added to the cavity of the tube, and/or be included in one or more layers formed from the alginate gel.

In an aspect of the method of forming multi-layered hollow alginate tubes for drug delivery, a 2-4% sodium alginate composition is dissolved in deionized water or a methanol/water mixture wherein the amount of methanol is 5% or less v/v to obtain a gel.

In an aspect of the invention, the open-ends of the tubes may be closed with an alginate cap. The drug delivery system comprises single layer alginate tubes, multi-layer alginate tubes or both. In some embodiments, the therapeutic drug is included in one or more layers of the alginate tubes. In other embodiments the therapeutic drug is not included in the alginate gel mixture and is placed in the cavity of the alginate tube. In other embodiments, the therapeutic drug is found in one or more layers of alginate and in the cavity of the alginate tube. In still other embodiments, two or more therapeutic drugs are present in the same or different layers of alginate. Two or more therapeutic drugs can be present in the cavity or one or more therapeutic drugs is present in one or more layers of alginate and one or more different therapeutic drugs are present in the cavity of the tube.

Water soluble or non-water soluble drugs can be used. The drug may be active in the stomach, intestine or colon. The alginate tube drug delivery system is particularly advantageous for delivery of drugs with poor solubility that require small doses, such as Glibenclamide or Furosemide. The alginate tubes are biodegradable and are quickly and easily excreted by the patient once the drug is released. The therapeutic drug may also be a drug that is absorbed in the proximal part of the gastrointestinal tract. The therapeutic drug may be a drug that is less soluble in alkaline pH. Non-limiting examples of drugs that can be used include Metformin HCL, Riboflavin, Ciprofloxacin HCl, Levodopa, Furosemide, Diazepam, Verapamil and Glibenclamide. Additional non-limiting examples of therapeutic drugs include:

Antipsychotics/CNS acting class of drugs including, but not limited to, carbidopa, chlordiazepoxide HCl, diazepam, haloperidol;

Antidiabetic drugs including, but not limited to, Remogliflozin etabonate, repaglinide, glyburide and other antidiabetic agents having poor solubility.

Antimicrobial drugs including, but not limited to, Ampicillin, Amoxicillin trihydrate;

Cardiovascular drugs including, but not limited to, atenelol, metprolol, captopril, -atenolol, sotalol;

Prokinetic agents including, but not limited to, Cisapride, metoclopramide, mosapride, and ferrous sulphate;

H2 Betablockers, including, but not limited to, Ranitidine, cimetidine, famotidine, nizatidine.

Pharmaceutically acceptable excipients may be added to the drug.

The hollow alginate tubes may be coated in order to delay the release of the drug and lower the concentration of drug released. The alginate tubes may be coated with a biodegradable polymer such as poly(lactic-co-glycolic acid) which is commonly known as “PLGA.” or other coatings known in the art such as Eudragit.

The alginate tube drug delivery system exhibits enhanced buoyancy in the stomach. This results in the alginate tube drug delivery system floating within the stomach for a longer period of time than if the drug itself was administered orally.

Another aspect of the alginate drug delivery system is that its activity is highly adjustable so that the release of the drug may be controlled as required to treat a specific patient or illness. The rate at which the system releases the drug and the concentration of the drug released can be adjusted by varying; the number of layers of the alginate tubes, the number of open or closed ends of the tubes, or the number of tube layers containing the drug. The hollow tubes may be filled with the drug in for example powder form, beads, microspheres, liquid etc to vary the concentration and rate of release of the therapeutic drug.

The alginate tube drug delivery system is adapted so that the rate of release of the therapeutic drug and the concentration level of the therapeutic drug can be adjusted to administer a drug in the stomach as required for specific treatments. The therapeutically optimal controlled drug release cycle will vary with the age and physical condition of the end user, the severity of the condition being treated, the duration of the treatment, the nature of concurrent therapy, the specific therapeutic drug employed, and like factors. The alginate tube drug delivery system is adapted to provide a wide range of controlled drug release cycles.

The drug delivery system is administered to the subject in need of the treatment or for prophylactic purposes. The drug delivery system can be administered orally. In certain embodiments it can be implanted into the gastrointestinal system or be incorporated into a suppository.

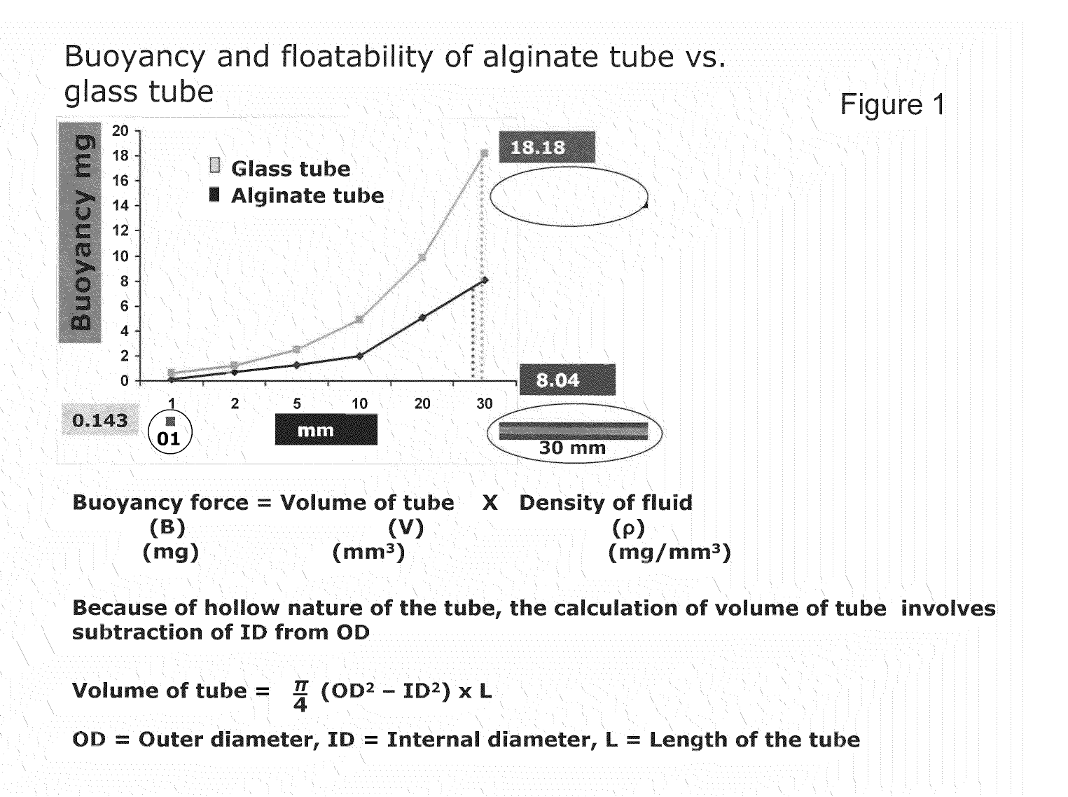

The alginate drug delivery system exhibits enhanced buoyancy in the stomach and the tubes can float within the stomach for long periods of time. As shown in FIG. 1, the buoyancy and floatability of a hollow alginate tube increases as the length of the alginate tube increases. At 30 mm, the buoyancy is about 8 times the buoyancy of a 2 mm alginate tube. As can be seen in the graph in FIG. 1, while the alginate tubes exhibit a high buoyancy force for a drug delivery system, the buoyancy force is less than that of a glass tube. The enhanced buoyancy increases the duration of the alginate tubes in the patient's stomach thereby providing an increased amount of drug absorption by the patient.

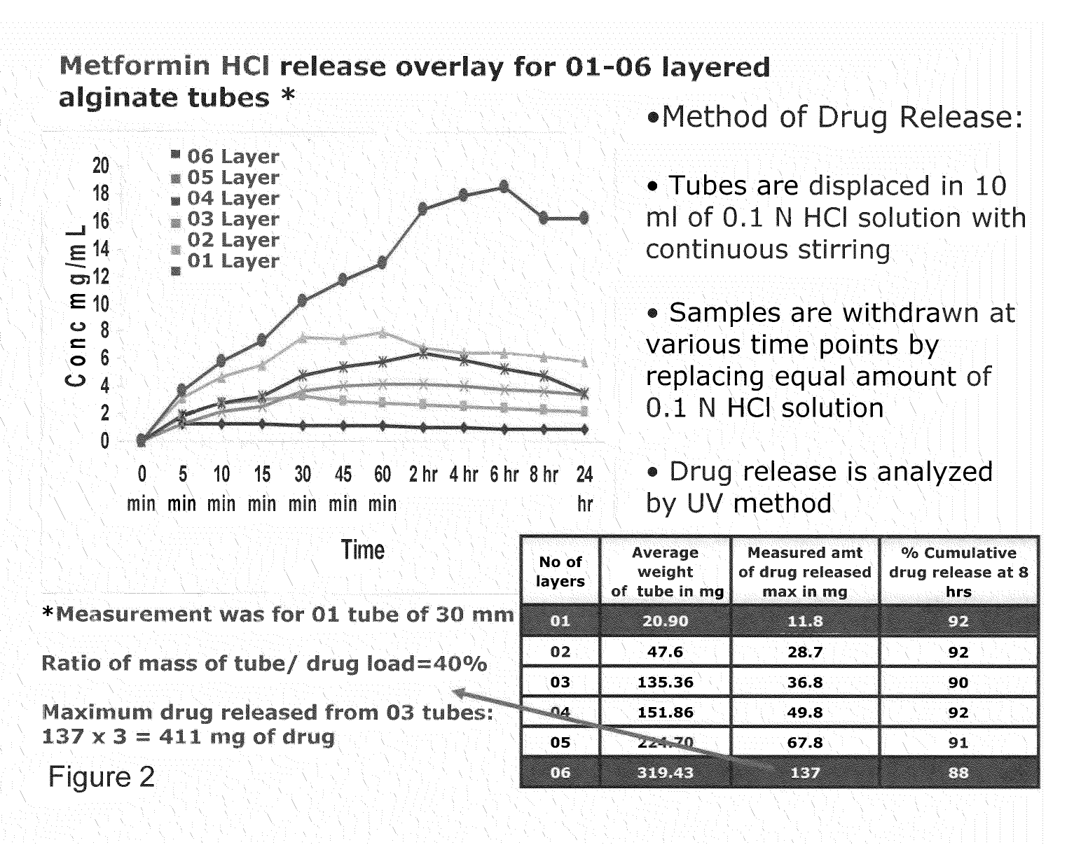

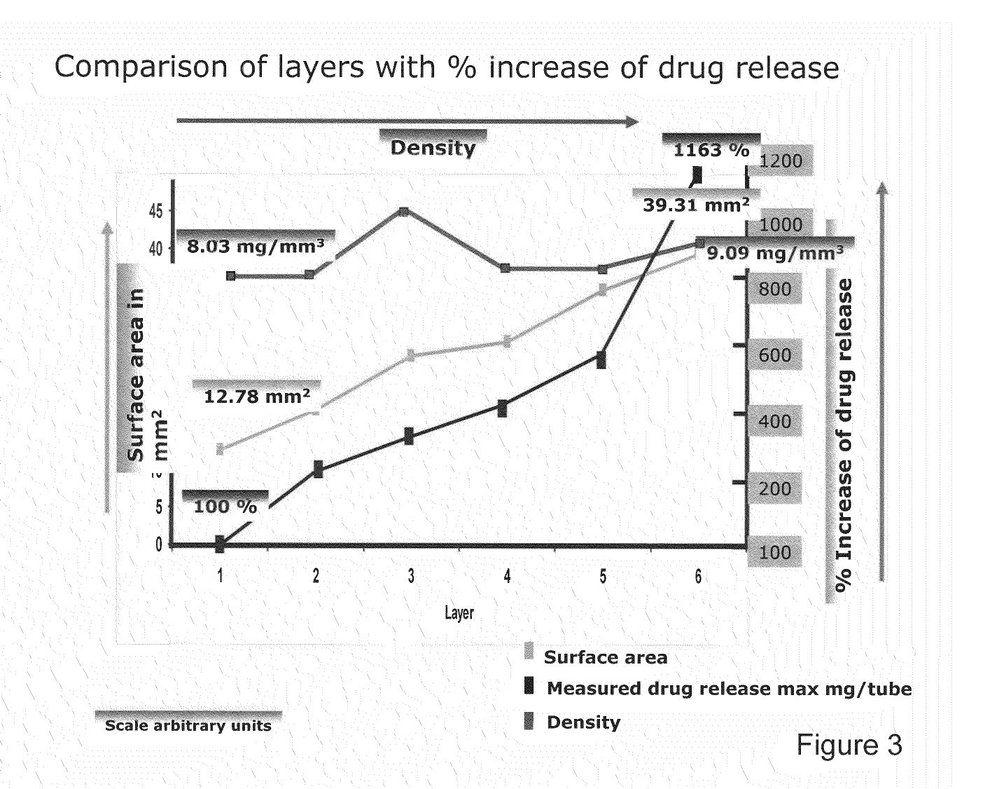

The number of layers of alginate tubes for the drug delivery system can be adjusted as necessary in order to adjust the concentration and rate of release of the therapeutic drug. The drug delivery system comprises between 1-6 layers of alginate. As shown in FIG. 2, the concentration and rate of Metformin HCl released in alginate tubes having the drug in the alginate layers is measured when the drug delivery system is comprised of 1-6 layers. As the results indicate, the drug delivery system delivers a greater concentration of drug as the number of layers increase. The concentration at which the six-layer system delivers the drugs increases significantly from 1 hour to 24 hours whereas the systems with 1 to 5 layers do not show an increase in drug release during this time period. In FIG. 3, the concentration of drug released in 1-6 layer systems is measured. The 1-5 layer systems increase relatively proportional to the number of layers in the drug delivery system. However, the 6-layer system having the drug in the alginate layers shows a 1163% increase of drug release which is significantly greater than systems with 5 or less layers having the drug in the alginate layers. This is due to increase in total surface area by addition of the sixth layer, compared to diameter/surface volume/area of the fifth layer. For instance, the concentration of drug released for the 6-layer system is approximately double the drug released by the 5-layer system. Accordingly, a drug delivery system comprising six layers of hollow alginate tubes having the drug in the alginate layers may be employed for treatments requiring high levels of a drug at a sustained rate.

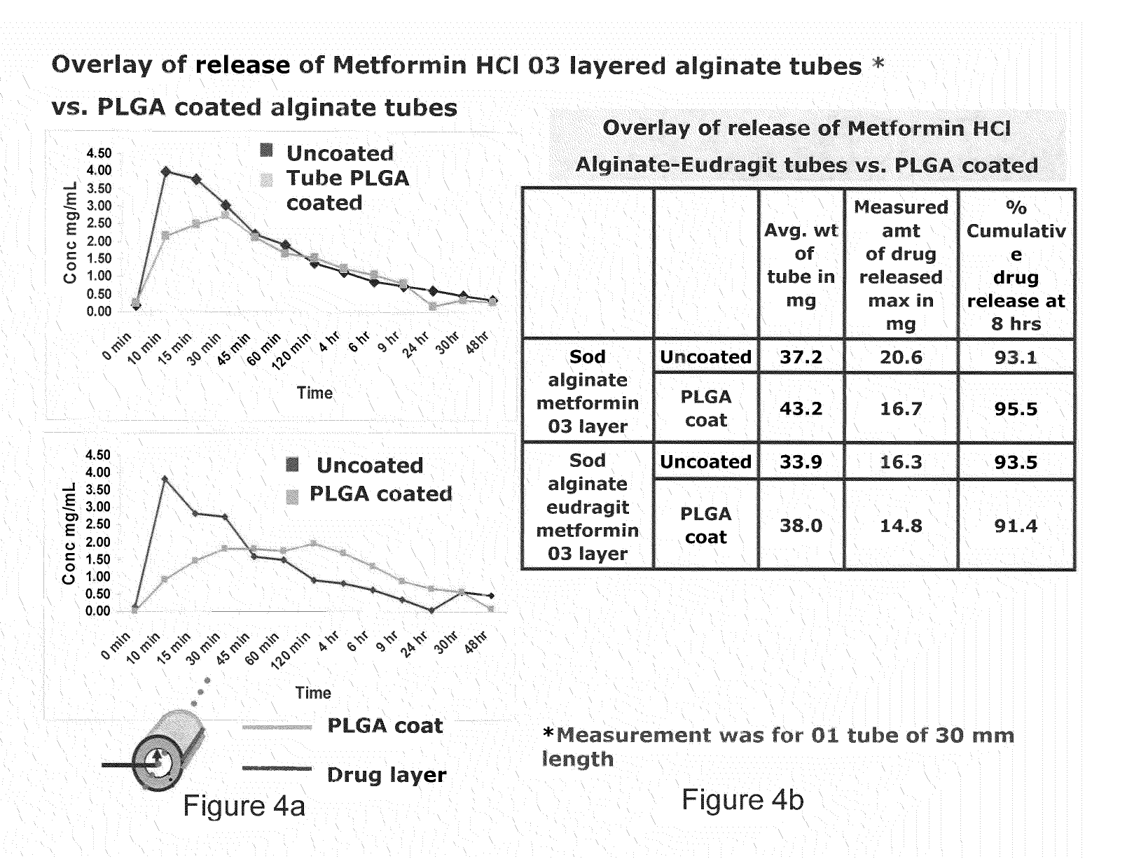

As shown in FIG. 4a, the concentration and rate of drug release of Metformin HCl is compared in a three-layered alginate tube system coated with PLGA and in the same system in an uncoated form. The uncoated alginate tubes were released at a much greater rate in the first 10 minutes and tapered off rapidly thereafter. In contrast, the PLGA coated alginate tubes released Metformin HCl more gradually during this time frame. While the uncoated alginate tubes released the Metformin HCl more rapidly for the first 30 minutes, after 30 minutes, the concentration of Metformin HCl released was approximately equal in the coated and uncoated alginate tube systems. Similarly, as shown in FIG. 4b, alginate eudragit tubes released Metformin HCl more gradually and in a lesser overall amount in coated form as compared to uncoated form. The PLGA coating comparatively lowers the burst release of the drug and the release rate of the drug is more controlled.

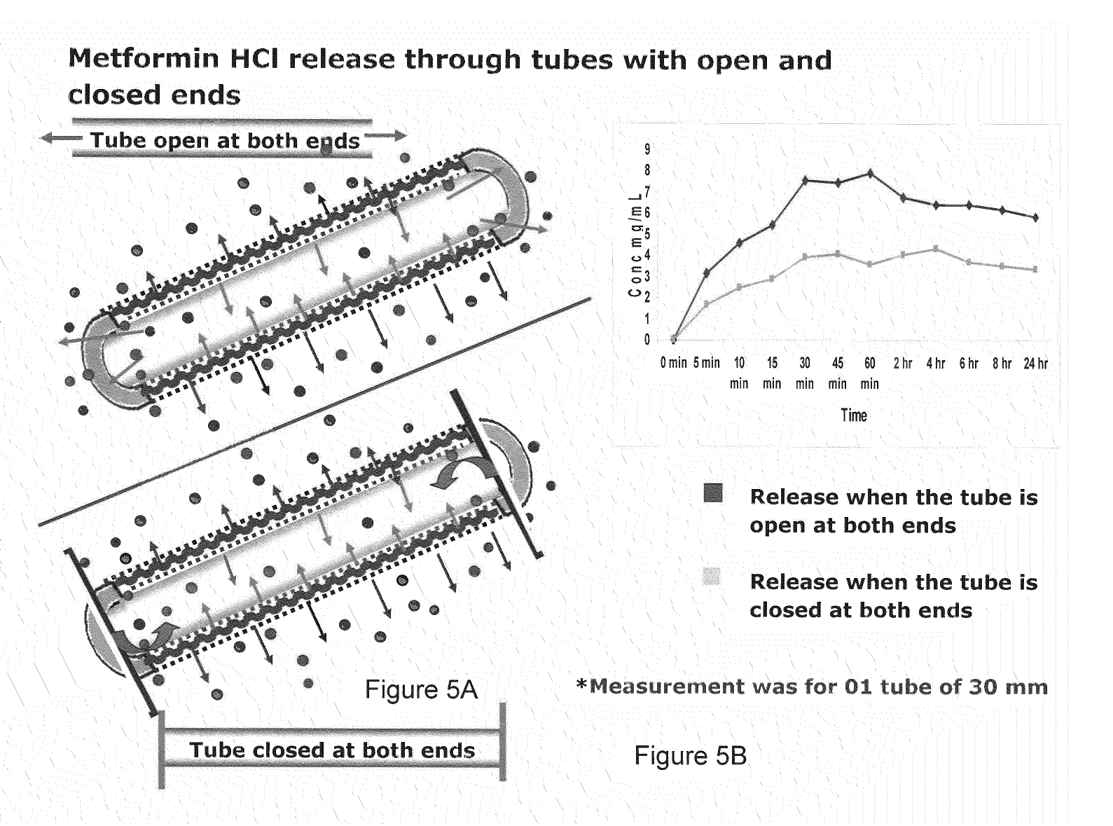

The release of the drug by the hollow alginate tube system can also be adjusted by forming open-ended tubes for one or more tubes of the system. Generally, a tube having open ends will release drugs more rapidly than a closed ended tube. As shown in FIGS. 5a and 5b the drug release of a system comprising one alginate tube was compared in embodiments having both ends opened and both ends closed. The rates at which the drug is released over the course of 24 hours change in similar patterns. However, the open-ended tube system exhibits a significantly higher rate of drug release that is at least double that of the closed ended tube system throughout the drug release cycle.

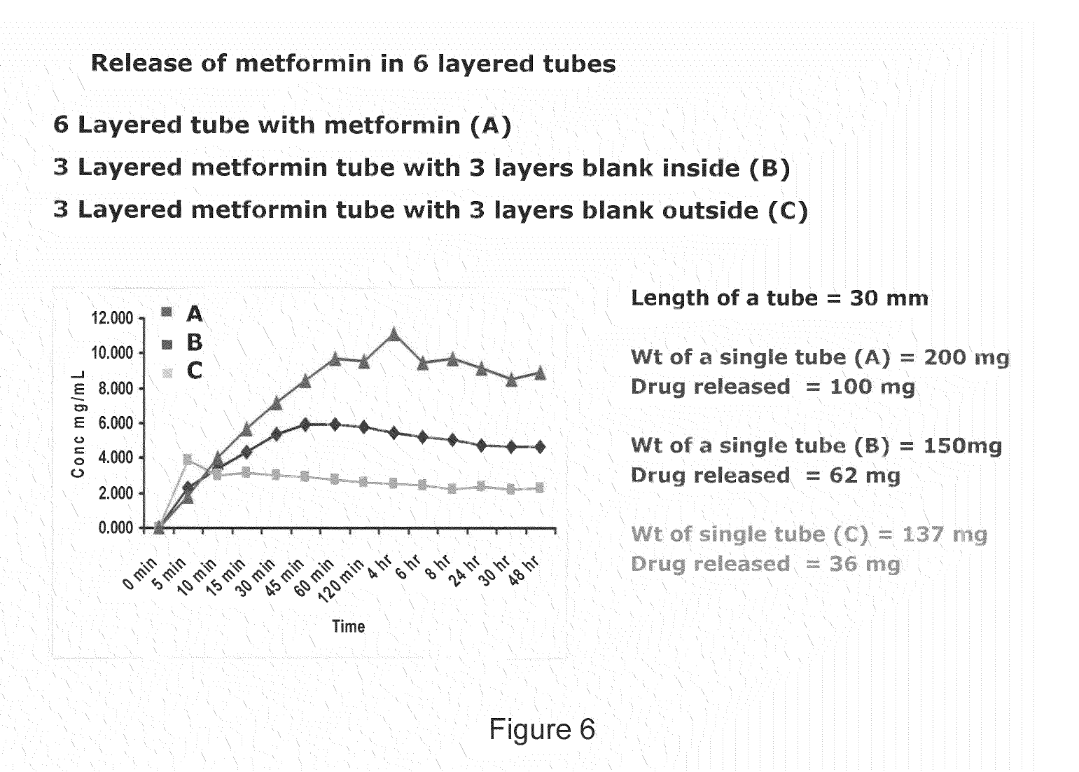

In order to optimize the concentration of the released drug and the rate at which the drug is released it may be preferred to include one or more layers that are formed from an alginate gel mixture but do not contain the therapeutic drug. For example, the rate of release could be delayed by adding layers that do not include therapeutic drug. For example, in case of a tube of six layers, wherein the inside two layers do not include drug and only two layers in the middle possess a drug, and next two top layers do not include drug, the rate of drug release from middle two layers will be comparatively slower than if the drug was included in the outside layers. As shown in FIG. 6, the multi-layered hollow alginate tube delivery systems comprised of six 30 mm tubes having three layers without drug release a much lower concentration of Metformin HCl than the system with six 30 mm tube layers having the therapeutic drug in each layer. The drug release system having the three outermost layers without the drug released about twice the concentration of Metformin HCl at five minutes as compared with the same drug release system having three inner layers without drug. From 10 minutes to approximately 48 hours the drug release system having three inner layers without the drug released Metformin HCl at a significantly higher level than the same system having three outermost layers without the drug.

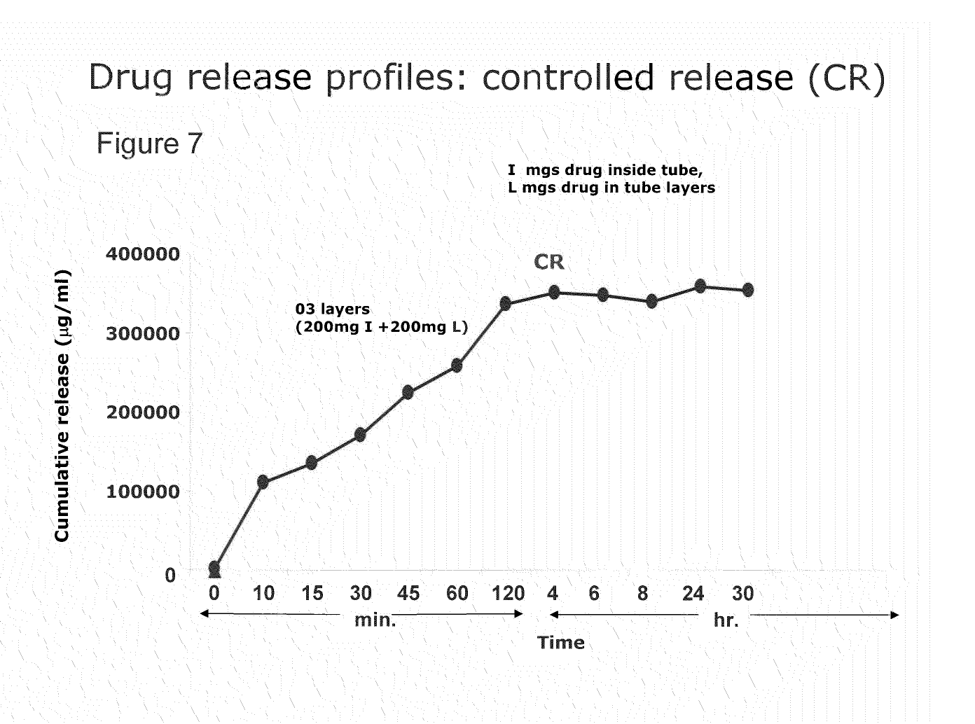

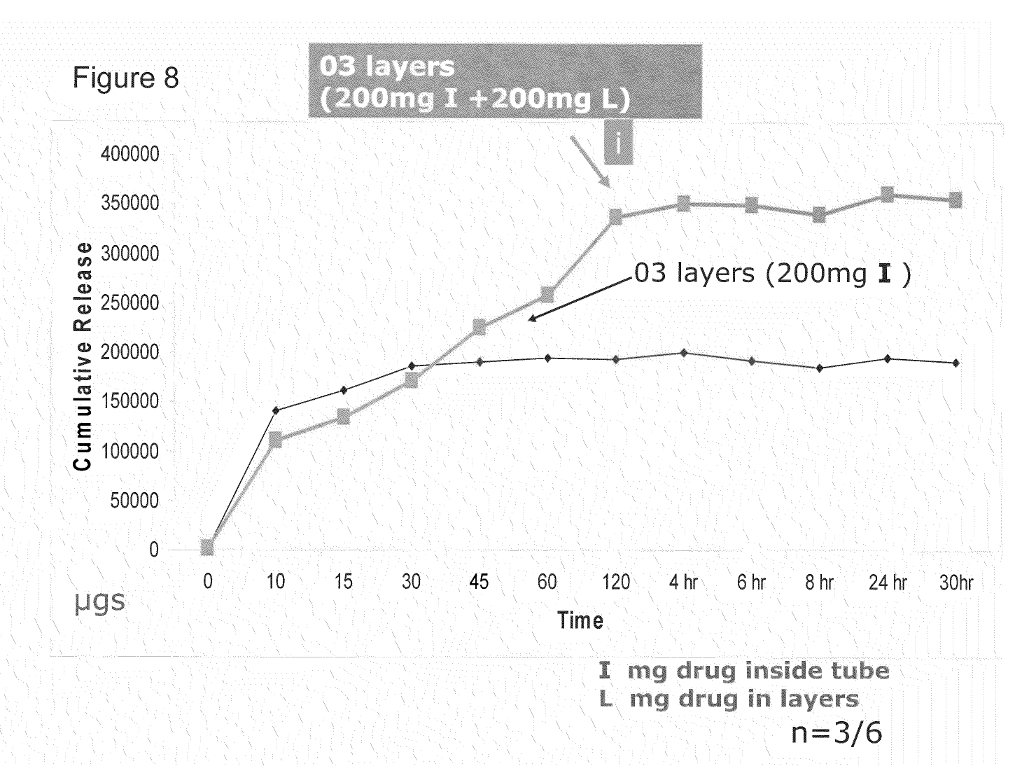

In another aspect of the invention, therapeutic drug is loaded into the cavity of an alginate tube. In an aspect of the invention, a powder form of the drug is placed in the (central) cavity of the alginate tube. The alginate tube is then closed off with an alginate cap. As shown in FIG. 7, a three-layered alginate tube delivery system contains 200 mg of the therapeutic drug in each of the layers of the alginate tubes. An additional 200 mg of the therapeutic drug is placed within each of the cavities of the alginate tubes. The concentration of the release increases steadily from 0 to 120 minutes. At approximately 100 minutes the drug release levels off at 350000 ug/ml and remains at this level with little fluctuation from 100 minutes to 30 hours. The drug release rate is measured by withdrawing the samples and analyzing it by UV spectroscopy. The sink volume is maintained by replacing equivalent amount of media. The concentration of drug can be a measure of actual release of the drug or as a cumulative release at particular time point. This constitutes an extremely consistent and sustained release of the drug. As shown in FIG. 8, after 30 minutes the three-layered alginate tube delivery system having 200 mg of the therapeutic drug in each of the tube outer layers of the bodies and an additional 200 mg of the powdered drug in each of the tube cavities releases a greater amount of the therapeutic drug than the delivery system having a three-layered blank alginate tubes having 200 mg of the therapeutic drug inside each of the tube cavities. From approximately 100 minutes to 30 hours the level of therapeutic drug released by the three-layered alginate tube delivery system having 200 mg of the therapeutic drug in each of the alginate layers and an additional 200 mg of the powdered drug in each of the tube cavities is roughly double the amount released by the alginate tubes with no drug in the three outer layers of alginate with 200 mg of the therapeutic drug inside each of the tube cavities.

The drug delivery system according to this invention can comprise one or more of the alginate tubes described above and the alginate tubes can be the same or different. For example, some of the alginate tubes may contain drug in each layer and others may contain drug in only certain layers or in cavity. It is also not required that the drug be the same in each tube in the system.

It is understood that modifications that do not substantially affect the activity of the various embodiments of this invention are included within the invention disclosed herein. Accordingly, the following examples are intended to illustrate but not to limit the present invention.

### EXAMPLES

### Example 1

### Preparation of Alginate Gel and Process of Layering it on Glass Capillary Tubes

Sodium alginate (3% w/w) was prepared in distilled water to get a desired viscosity, enough for pouring the viscous blend from the beaker. The glass capillary is dipped into the gel for 5-6 seconds and removed slowly such that the viscous gel is coated on to the surface of the glass capillary and immediately immersed into calcium chloride solution (5% w/w). The viscous blend immediately gets converted into solid mass in the form of tube along the surface of glass capillary which can be removed by slight pressure and sliding the tube along horizontal axis of the glass capillary. The tube is then air dried. The second and the subsequent layers till six layers are deposited by the same method as mentioned above.

### Example 2

### Method of Dug Release Studies

Drug release studies were carried out in 10 mL volume of media (0.1N HCl). Three tubes of equal length (3 cm) were weighed and kept in 0.1N HCl (mimicking the Gastro simulated fluid) for release, the study was conducted in triplicate, aliquots of 1 ML was withdrawn at an interval of 5 min, 10 min, 15 min, 30 min, 45 min, 60 min, 120 min, 4 hr, 6 hr, 8 hr, 24 hr, 30 hr, 48 hr and filtered through Whatman filter paper to reduce the interference by alginate polymer. Concentration was determined by UV-Vis spectrophotometer with suitable dilution if needed. The calibration curve for standard Metformin hydrochloride was prepared in 0.1N HCl at wavelength maxima of 235 nm.

What is claimed is:

1. A controlled release gastro-retentive drug delivery system comprising at least one tube having one or more layers of alginate and a therapeutic drug.

2. The drug delivery system according to claim 1 wherein the therapeutic drug is present in one or more layers of alginate.

3. The drug delivery system of claim 1 wherein the therapeutic drug is present in a cavity of at least one tube.

4. The drug delivery system of claim 3 wherein in at least one alginate tube the therapeutic drug is present in one or more alginate layers and in the cavity of the tube.

5. The drug delivery system of claim 3 wherein in at least one alginate tube one or more therapeutic drugs are present in one or more alginate layers and one or more of the same or different therapeutic drugs are present in a cavity of the tube.

6. The drug delivery system of claim 1 wherein the drug delivery system has a plurality of therapeutic drugs.

7. The drug delivery system of claim 1 wherein at least one alginate tube is hollow and has open ends.

8. The drug delivery system of claim 1 wherein at least one alginate tube is hollow and has at least one closed end.

9. The drug delivery system of claim 1 wherein at least one tube has a coating other than alginate covering the exterior of said tube.

10. A method for manufacturing an alginate tube for a drug delivery system, comprising the steps of:
a) coating a substrate with an alginate gel;
b) reacting the coated substrate with a salt;
c) drying the coated substrate; and
d) removing the coating from the substrate whereby the coating forms a drug delivering body.

11. The method of claim 10 wherein a therapeutic drug is included in the alginate gel.

12. The method of claim 10 comprising adding a therapeutic drug in a cavity of the alginate tube.

13. The method of claim 10 comprising adding one or more additional layers of alginate.

14. The method of claim 10 wherein the alginate tube has between one and six layers.

15. The method of claim 10 wherein one or both ends of at least one alginate tube is capped to form closed ends.

16. The method of claim 10 comprising the further step of covering the exterior of the alginate tubes with a coating other than alginate.

17. A method for treatment of an illness in a subject or treatment for prophylactic purposes comprising the step of orally administering a drug delivery system comprising at least one tube having at least one layer of alginate and a therapeutic drug to the subject in need thereof.
